(19) United States
(12) Patent Application Publication
(10) Pub. No.: US 2022/0184593 A1
(43) Pub. Date: Jun. 16, 2022

(54) A PROCESS FOR THE PRODUCTION OF A CATALYST, A CATALYST THEREFROM AND A PROCESS FOR PRODUCTION OF ETHYLENICALLY UNSATURATED CARBOXYLIC ACIDS OR ESTERS

(71) Applicant: MITSUBISHI CHEMICAL UK LIMITED, Billingham (GB)

(72) Inventors: Adam CULLEN, Wilton, Redcar (GB); Wataru Ninomiya, Otake-shi (JP)

(21) Appl. No.: 17/438,612

(22) PCT Filed: Mar. 13, 2020
(86) PCT No.: PCT/GB2020/050644

§ 371 Date: Sep. 13, 2021

(30) Foreign Application Priority Data
Mar. 13, 2019 (GB) 1903455.2

(51) Int. Cl. B01J 37/02; B01J 21/08; B01J 35/10; B01J 37/08

(52) U.S. Cl. CPC B01J 37/0207; B01J 21/08; B01J 6/001; B01J 35/1042; B01J 37/08; B01J 35/1038

(57) ABSTRACT

A process for producing a catalyst including a) providing an uncalcined metal modified porous silica support wherein the modifier metal is selected from one or more of boron, magnesium, aluminium, zirconium, hafnium and titanium, wherein the modifier metal is present in mono- or dinuclear modifier metal moieties; b) optionally removing any solvent or liquid carrier from the modified silica support; c) optionally drying the modified silica support; d) treating the uncalcined metal modified silica support with a catalytic metal to effect adsorption of the catalytic metal onto the metal modified silica support; and e) calcining the impregnated silica support of step d). The invention extends to an uncalcined catalyst intermediate and a method of producing a catalyst by providing a porous silica support having isolated silanol groups.

The present invention relates to a process for producing a modified silica catalyst, the catalyst and a process for the production of ethylenically unsaturated carboxylic acids or esters, particularly α, β unsaturated carboxylic acids or esters, more particularly acrylic acids or esters such as (alk)acrylic acids or alkyl (alk)acrylates especially (meth)acrylic acids or alkyl (meth)acrylates such as methacrylic acid (MAA) and methyl methacrylate (MMA) by the condensation of carboxylic acid or esters with formaldehyde or a source thereof such as dimethoxymethane in the presence of such catalysts, in particular, by the condensation of propionic acid or alkyl esters thereof such as methyl propionate with formaldehyde or a source thereof in the presence of such catalysts. The invention is therefore particularly relevant to the production of MAA and MMA. The catalysts of the present invention incorporate a modified silica support uniquely modified by a particular modifier metal and a catalytic metal.

As mentioned above, the unsaturated acids or esters may be made by the reaction of a carboxylic acid or ester and suitable carboxylic acids or esters are alkanoic acids (or esters) of the formula R3-CH2-COOR4, where R3 and R4 are each, independently, a suitable substituent known in the art of acrylic compounds such as hydrogen or an alkyl group, especially a lower alkyl group containing, for example, 1-4 carbon atoms. Thus, for instance, MAA or alkyl esters thereof, especially MMA, may be made by the catalytic reaction of propionic acid, or the corresponding alkyl ester, e.g. methyl propionate, with formaldehyde as a methylene source in accordance with the reaction sequence 1.

R³—CH₂—COOR⁴+HCHO------->R³—CH(CH₂OH)—COOR⁴

and

R³—CH(CH₂OH)—COOR⁴------>R³—C(:CH₂)—COOR⁴+H₂O Sequence 1

An example of reaction sequence 1 is reaction sequence 2

CH₃—CH2—COOR⁴+HCHO------->CH₃—CH(CH₂OH)—COOR⁴

CH₃—CH(CH₂OH)—COOR⁴------>CH₃—C(:CH₂)—COOR⁴+H₂O Sequence 2

The above reaction sequences are typically effected at an elevated temperature, usually in the range 250-400° C., using an acid/base catalyst. Where the desired product is an ester, the reaction is typically effected in the presence of the relevant alcohol in order to minimise the formation of the corresponding acid through hydrolysis of the ester. Also for convenience it is often desirable to introduce the formaldehyde in the form of a complex of formaldehyde with methanol. Hence, for the production of MMA, the reaction mixture fed to the catalyst will generally consist of methyl propionate (MEP), methanol, formaldehyde and water.

A known production method for MMA is the catalytic conversion of MEP to MMA using formaldehyde. A known catalyst for this is a caesium catalyst incorporating a support, for instance, silica.

WO99/52628 discloses preparation of a modifier metal (boron, magnesium, aluminium, zirconium and hafnium) impregnated catalyst from a mesoporous gel silica using modifier nitrates, oxynitrates and oxides such as zirconium nitrate followed by caesium carbonate incorporation and calcining. Zirconium or zirconium and aluminium acetate solution is mixed with caesium acetate solution and adsorbed together onto the silica support.

U.S. Pat. No. 6,887,822 teaches the option of calcining a hydrogel silica surface after treatment with a catalytic metal. However, it does not address the issue of adsorption of modifier metals and how to treat a surface so modified. Instead, zirconia is introduced by co-gelation. The document teaches that silica xerogel bead impregnation is precluded and only hydrogel beads are exemplified apparently leading to much stronger beads.

Unpublished application PCT/GB2018/052606 discloses adsorption of metal organic complexes of zirconium and hafnium onto silica supports followed by adsorption of catalytic metal such as caesium. Generally, a calcination step after modifier metal adsorption is taught especially where the modifier is added as a complex as well as an optional calcination step after alkali metal adsorption.

Generally, after treatment of a silica support with modifier metals a calcination step to “fix” the metal prior to further treatment would be expected. This is particularly the case when organic groups are attached to the modifier metals and need to be removed.

The present inventors have now discovered that catalysts produced by the invention provide a high level of selectivity in the condensation of methylene sources such as formaldehyde with a carboxylic acid or alkyl ester such as MEP.

Still further, the present inventors have found that when the process of catalyst production of the invention is used, the rate of catalyst surface sintering has been found to be retarded and loss of surface area upon which the catalytic reaction takes place during the condensation reaction is reduced.

Therefore, the catalysts of the invention are remarkably effective catalysts for the production of α, β ethylenically unsaturated carboxylic acids or esters by condensation of the corresponding acid or ester with a methylene source such as formaldehyde providing several advantages such as high levels of selectivity and/or reduced sintering of the catalyst surface.

According to a first aspect of the present invention there is provided a process for producing a catalyst comprising the steps of:

a) providing an uncalcined metal modified porous silica support wherein the modifier metal is selected from one or more of B, Mg, Al, Zr, Hf and Ti, wherein the modifier metal is present in mono- or dinuclear modifier metal moieties;
b) optionally, removing any solvent or liquid carrier from the modified silica support
c) optionally, drying the modified silica support
d) treating the uncalcined metal modified silica support with a catalytic metal to effect adsorption of the catalytic metal onto the metal modified silica support and
e) calcining the impregnated silica support of step d).

Advantageously, by treating the uncalcined modified silica support as defined with catalytic metal followed by subsequent calcination, an improved selectivity and increased resistance to sintering is found in the catalytic production of ethylenically unsaturated carboxylic acids or esters by the condensation of carboxylic acid or esters with formaldehyde or a source thereof.

In the present invention, it has been found that controlling the nuclearity of the modifier metal moieties is surprisingly advantageous because it controls the proximity of neighbouring modifier metal moieties on the silica.

According to a second aspect of the present invention there is provided an uncalcined catalyst intermediate comprising an uncalcined porous silica support modified with a modifier metal wherein the modifier metal is selected from one or more of B, Mg, Al, Zr, Hf and Ti, wherein the said modifier metal is present in mono- or dinuclear modifier metal moieties and catalytic metal adsorbed on the said uncalcined modified silica support.

The silica of the first or second aspect may be provided as a co-gel of the modifier metal oxide and silica or as a modified silica with the modifier metal adsorbed on the silica surface.

Surprisingly, the catalyst of the present invention provides improved selectivity and increased resistance to sintering.

Surprisingly, it has been found that increasing the temperature of calcination provides further improved selectivity.

According to a third aspect of the present invention there is provided a catalyst obtained by a process of the first or further aspect of the present invention.

According to a fourth aspect of the present invention there is provided a catalyst obtainable by the process of the first or further aspect of the present invention.

According to further aspects of the present invention there is provided methods of producing modified silica supports for a catalyst or catalysts according to the claims.

### Modifier Metal Complex

Typically, when the modifier metal is added as an adsorbate it may be added as a mono- or dinuclear modifier metal compound. Typically, the compound is a complex and the ligands in the coordination sphere of the compound are generally of sufficient size to prevent further oligomerisation of the modifier metal, and/or significant increase in nuclearity of the complex, prior to and/or after adsorption. Generally, increase in nuclearity to dimers may be acceptable. Typically, the modifier metal complex is an organic complex with one or more organic polydentate chelating ligands, or alternatively a complex with sterically bulky monodentate ligands effective to stabilise the nuclearity.

Typically, at least 25%, of the said modifier metal either before or after calcination is present on the support in the form of mono- or dinuclear modifier moieties. Accordingly, typically, at least 25%, of the said modifier metal is present on the support in the form of modifier metal moieties derived from a mono- or dinuclear metal compounds.

Typically, the mono- or dinuclear modifier metal contacts the silica support as a mono- or dinuclear modifier metal compound in solution to effect adsorption of the said modifier metal onto the support.

Typically, the modifier metal compound is mononuclear or dinuclear, more preferably, mononuclear.

Clusters of modifier metal of more than 2 metal atoms dispersed throughout the support such as a hydrogel support, have surprisingly been found to decrease reaction selectivity for the production of α, β ethylenically unsaturated carboxylic acids or esters by condensation of the corresponding acid or ester with a methylene source such as formaldehyde. Such large clusters have also surprisingly been found to increase sintering of the modified silica particles relative to mononuclear or dinuclear moieties thereby reducing the surface area which lowers strength and reduces the life of the catalyst before activity becomes unacceptably low. In addition, selectivity is often lower, depending on the nature of the cluster of the modifier metal.

Advantageously, when at least a proportion of the modifier metal incorporated into the modified silica of the above aspects of the present invention is derived from a mono- or dinuclear modifier metal cation source at the commencement of the modified silica formation, there has been found to be improved reaction selectivity and/or reduced rate of sintering of the catalyst surface during the production of α, β ethylenically unsaturated carboxylic acids or esters.

Typically, the modifier metal is selected from zirconium, hafnium and titanium.

Typically, the metal compound is a complex which comprises two or more chelating ligands, preferably, 2, 3 or 4 chelating ligands. The chelating ligands herein may be bi, tri, tetra or polydentate. However, it is also possible for the compound to include bulky monodentate ligands which are also effective to effectively space as set out herein the modifier metals on the silica surface.

Typically, the metal complex is tetracoordinate, pentacoordinate, hexacoordinate, heptacoordinate, or octacoordinate.

Advantageously, the size of the ligands in the coordination sphere of the metal compound such as the size of the chelating ligands causes the modifier metal to be more disperse than the same modifier metal with a simple counterion such as nitrate, acetate or oxynitrate. It has been found that smaller metal salt adsorption leads to clustering of the modifier metal following heat treatment or calcination which in turn lowers the selectivity of the catalyst and lowers sintering resistance of the catalyst.

Generally, herein the modifier metal is an adsorbate adsorbed on the silica support surface of the catalyst. The adsorbate may be chemisorbed or physisorbed onto the silica support surface as its compound, typically, it is chemisorbed thereon.

Suitable chelating ligands herein may be non-labile ligands optionally selected from molecules with lone pair containing oxygen or nitrogen atoms able to form 5 or 6 membered rings with a modifier metal atom. Examples include diones, diimines, diamines, diols, dicarboxylic acids or derivatives thereof such as esters, or molecules having two different such functional groups and in either case with the respective N or O and N or O atom separated by 2 or 3 atoms to thereby form the 5 or 6 membered ring. Examples include pentane-2,4-dione, esters of 3-oxobutanoic acid with aliphatic alcohols containing 1-4 carbon atoms such as ethyl 3-oxobutanoate, propyl 3-oxobutanoate, isopropyl 3-oxobutanoate, n-butyl 3-oxobutanoate, t-butyl 3-oxobutanoate, heptane-3,5-dione, 2,2,6,6,-Tetramethyl-3,5-heptanedione, 1,2-ethanediol, 1,2-propanediol, 1,3-propanediol, 1,3-butanediol, 1,2-butanediol, 1,2-diaminoethane, ethanolamine, 1,2-diamino-1,1,2,2-tetracarboxylate, 2,3-dihydroxy-1,4-butanedioate, 2,4-dihydroxy-1,5-pentanedioate, salts of 1,2-dihydroxylbenzene-3-5-disulphonate, diethylenetriaminepentaacetic acid, nitrolotriacetic acid, N-hydroxyethylethylenediaminetriacetic acid, N-hydroxyethyliminodiacetic acid, N,N-dihydroxyethylglycine, oxalic acid and its salts. Pentane-2,4-dione, heptane-3,5-dione, 2,2,6,6-Tetramethyl-3,5-heptanedione, ethyl 3-oxobutanoate and t-butyl 3-oxobutanoate are most preferred. The smaller bidentate chelating ligands having, for example less than 10 carbon and/or hetero atoms in total enable small complexes to be formed which can allow higher concentrations to be deposited on the surface of the silica compared to larger ligands. Accordingly, the mononuclear or dinuclear modifier metal cation source herein may be in the form of complexes of modifier metal with such smaller chelating ligands, preferably, with at least one such ligand. Such compounds may include labile ligands such as solvent ligands, for example in alcohol solvent, alkoxide ligands such as ethoxide or propoxide etc.

The chelating ligand is typically a non-labile ligand. By non-labile ligand is meant a ligand that is co-ordinated to the modifier metal and is not removed by the adsorption of the modifier metal onto the silica surface. Accordingly, the non-labile ligand is typically coordinated to the modifier metal in solution prior to treatment of the silica surface with modifier metal. For the avoidance of doubt, the non-labile ligand is typically removed by suitable treatment of the silica surface following adsorption of the modifier metal.

The size of the chelating ligands are selected so as to space the modifier metal atoms apart on the silica surface to prevent combination thereof during the catalyst production.

Alternatively, modifier metal complexes with bulky monodentate ligands—to prevent oligomerisation of the metal complexes—can be used. Typical ligands used in said complexes include, but are not limited to, alkoxides with suitable organic groups such as tert-butoxide or 2,6 di tert-butyl phenoxide, amides with suitable organic groups such as dialkylamides (methyl, ethyl and higher linear and branched alkyl groups, as well as bis (trimethylsilylamido) complexes, and alkyl ligands with suitable organic groups such as 2,2-dimethylpropyl (neopentyl) ligands.

Typically, the silica support has isolated silanol groups and by contacting the silica support with the modifier metal species, the modifier metal is adsorbed onto the surface of the silica support through reaction with said silanol groups.

Preferably, the adsorbed or co-gelated modifier metal cations are sufficiently spaced apart from each other by the modifier metal compound to substantially prevent oligomerisation thereof during subsequent treatment steps such as the impregnation of catalytic metal, or optionally, subsequent calcination, more preferably di, tri or oligomerisation thereof with neighbouring modifier metal cations.

Typically, at least 25%, more typically, at least 30%, such as at least 35%, more preferably at least 40%, such as at least 45%, most suitably at least 50%, such as at least 55%, for example at least 60% or 65%, and most preferably at least 70% such as at least 75% or 80%, more typically, at least 85%, most typically, at least 90%, especially, at least 95% of the said modifier metal species contacting the silica support in the contacting step are mononuclear and/or dinuclear species.

According to a fifth aspect of the present invention there is provided a method of producing a catalyst according to any of the aspects herein or otherwise comprising the steps of:
a) providing a porous silica support having isolated silanol groups;
b) treating the said porous silica support with mono- or dinuclear modifier metal compound so that modifier metal is adsorbed onto the surface of the silica support through reaction with said isolated silanol groups, wherein the adsorbed modifier metal atoms are sufficiently spaced apart from each other to substantially prevent oligomerisation thereof with neighbouring modifier metal atoms prior to and/or after calcination, more preferably, sufficiently spaced apart from each other to substantially prevent dimerisation or trimerisation thereof with neighbouring modifier metal atoms thereof wherein the modifier metal is selected from B, Mg, Al, Zr, Hf and Ti;
c) optionally removing any solvent or liquid carrier from the modified silica support
d) optionally drying the modified silica support
e) treating the uncalcined modified silica support with a catalytic alkali metal to effect adsorption of the catalytic alkali metal onto the modified silica support; and
f) calcining the impregnated silica support of step e).

Preferably, the spacing apart of the modifier metal atoms is effected by the size of the modifier metal compound.

Typically, the silica support comprises isolated silanol groups (-SiOH) at a level of <2.5 groups per nm².

Preferably, the modifier metal herein is a solution of compounds of the said modifier metal so that the compounds are in solution when contacted with the support to effect adsorption onto the support.

Typically, the solvent for the said solution is water or other than water.

Typically, the solvent is an organic solvent such as toluene or heptane, Further, the solvent may be an aliphatic or aromatic solvent. Still further, the solvent may be a chlorinated solvent such as dichloromethane. More typically, the solvent is an aliphatic alcohol, typically selected from C1-C6 alkanols such as methanol, ethanol, propanol, isopropanol, butanols, pentanols and hexanols, more typically, methanol, ethanol or propanols.

The isolated silanol group concentration on the silica support prior to modifier metal adsorption is preferably controlled by calcination or other suitable methods as known to those skilled in the art. Methods of identification of silanols include for example L T Zhuravlev, in “Colloids and Surfaces: Physicochemical and Engineering Aspects, vol. 173, pp. 1-38, 2000” which describes four different forms of silanols: isolated silanols, geminal silanols, vicinal silanols, and internal silanols which can coexist on silica surfaces. Isolated silanol groups are most preferred. These can be identified by infrared spectroscopy as a narrow absorption peak at 3730-3750cm′ whereas other silanols display broad peaks between 3460 and 3715cm⁻¹ (see “The Surface Properties of Silicas, Edited by Andre P Legrand, John Wiley and Sons, 1998 (ISBN 0-471-95332-6) pp. 147-234).

The modified silica support according to any of the aspects herein may comprise isolated silanol groups (—SiOH) at a level of <2.5 groups per nm². Typically, the modified support comprises isolated silanol groups (—SiOH) at a level of >0.1 and <2.5 groups per nm², more preferably, at a level of from 0.2 to 2.2, most preferably, at a level of from 0.4 to 2.0 groups per nm².

Still further the invention extends to a process, catalyst or catalyst intermediate according to any aspects herein, wherein the support comprises the said modifier metal moieties present on the support and present at a level of <2.5.0 moieties per nm².

Typically, the support comprises the said modifier metal moieties at a level of >0.025 and <2.5 groups per nm², more preferably, at a level of from 0.05 to 1.5, most preferably, at a level of from 0.1 to 1.0 moieties per nm².

The concentration of preferably isolated silanol groups determines the maximum number of modifier metal can be effectively determined because the distribution of silanol sites will generally be uniform. The isolated silanol concentration for the production of a modified silica support according to the present invention may be below 2.5 groups per nm², more typically, less than 2.5 groups per nm², most typically, less than 1.5 groups per nm², especially, less than 0.8 groups per nm². Suitable ranges for the silanol concentration for production of a modified silica supports may be 0.1-4.6 silanol groups per nm², more preferably 0.15-2.5 silanol groups per nm², most preferably 0.2-1.0 silanol groups per nm2.

The concentration of the modifier metal complex, should be set at a level that prevents the significant formation of bilayers etc. on the surface of the support which would lead to modifier metal to metal interaction. In addition, filling in of gaps in the initial monolayer that could result in weak adsorption of the modifier metal away from isolated silanol sites should also be avoided to prevent interaction with neighbouring strongly adsorbed modifier metals. Typical concentration ranges for the modifier metals of the invention may be as set out herein.

Typically, at least 30% , such as at least 35%, more preferably at least 40%, such as at least 45%, most suitably at least 50%, such as at least 55%, for example at least 60% or 65%, and most preferably at least 70% such as at least 75% or 80%, more typically, at least 85%, most typically, at least 90%, especially, at least 95% of the modifier metal in the modifier metal complex are mononuclear and/or dinuclear modifier metal compounds when the complex is contacted with the support to effect adsorption of the said complex onto the support.

A suitable method of treating the silica to provide the isolated silanol groups at the level specified herein is by calcination. However, other techniques such as hydrothermal treatment or chemical dehydration are also possible. U.S. Pat. No. 5,583,085 teaches chemical dehydration of silica with dimethyl carbonate or ethylene dicarbonate in the presence of an amine base. U.S. Pat. Nos. 4,357,451 and 4,308,172 teach chemical dehydration by chlorination with SOCl₂ followed by dechlorination with H₂ or ROH followed by oxygen in a dry atmosphere. Chemical dehydration may provide up to 100% removal of silanols against a minimum of 0.7/nm² by thermal treatment. Thus, in some instances, chemical dehydration may provide more scope for silanol group control.

The term isolated silanol (also known as single silanol) is well known in the art and distinguishes the groups from vicinal or geminal or internal silanols. Suitable methods for determining the incidence of isolated silanols include surface sensitive infrared spectroscopy and ¹H NMR or ³¹Si NMR.

Preferably, the silica support is dried or calcined prior to treatment with the modifier metal.

### Silica

Typically, the modified silica support is a xerogel. The gel may also be a hydrogel or an aerogel.

The gel may also be a silica-modifier metal oxide co-gel. The silica gel may be formed by any of the various techniques known to those skilled in the art of gel formation such as mentioned herein. In this case, the modifier metal oxide may also be distributed through the matrix of the silica as well as the surface thereof. However, typically, the modified silica gels are produced by a suitable adsorption reaction. Adsorption of the relevant modifier metal compounds to a silica gel such as a silica xerogel to form modified silica gel having the relevant mono- or dinuclear modifier metal moieties is a suitable technique.

The silica may be in the form of a gel prior to treatment with the modifier metal adsorbate. The gel may be in the form of a hydrogel, a xerogel or an aerogel at the commencement of modification. Typically, the silica support is a hydrogel or xerogel, most preferably a xerogel.

As mentioned, methods for preparing silica gels are well known in the art and some such methods are described in The Chemistry of Silica: Solubility, Polymerisation, Colloid and Surface Properties and Biochemistry of Silica, by Ralph K Iler, 1979, John Wiley and Sons Inc., ISBN 0-471-02404-X and references therein.

The silica component of the modified silica support may typically form 80-99.9 wt % of the modified support, more typically 85-99.8 wt %, most typically 90-99.7 wt % thereof.

The porous silica support has typically a range of pore sizes between mesoporous and macroporous with an average pore size of between 2 and 1000 nm, more preferably between 3 and 500 nm, most preferably between 5 and 250 nm. Macropore size (above 50 nm) can be determined by mercury intrusion porosimetry using NIST standards whilst the Barrett-Joyner-Halenda (BJH) analysis method using liquid nitrogen at 77 K is used to determine the pore size of mesopores (2-50 nm). The average pore size is the pore volume weighted average of the pore volume vs. pore size distribution.

Surprisingly, it has also been found that preparing the modified silica support by co-gelation of a xerogel and then carrying out steps b) to e) of the first aspect of the present invention also results in a catalyst with improved selectivity and increased sintering resistance.

Still further, according to a sixth aspect of the present invention there is provided a catalyst comprising an intermediate according to the second aspect of the present invention, wherein the said uncalcined intermediate has been calcined.

### Catalytic Metal

Generally, herein the catalytic alkali metal is an adsorbate adsorbed on the modified silica support surface of the catalyst. The adsorbate may be chemisorbed or physisorbed onto the modified silica support surface, typically, it is chemisorbed thereon.

The catalytic metal herein is a metal other than modifier metal. Preferably, the catalytic metal may be selected from one or more alkali metals. Typically, the catalytic alkali metal is selected from caesium, potassium or rubidium, more preferably, caesium.

Suitably the catalytic metals such as caesium may be present in the catalyst at a level of at least 1 mol/100 (silicon+modifier metal) mol more preferably, at least 1.5 mol/100 (silicon+modifier metal) mol, most preferably, at least 2 mol/100 (silicon+modifier metal) mol. The level of catalytic metal may be up to 10 mol/100 (silicon+modifier metal) mol in the catalyst, more preferably, up to 7.5 mol/100 (silicon+modifier metal) mol, most preferably, up to 5 mol/100 (silicon+modifier metal) mol in the catalyst.

Preferably, the level of catalytic metal in the catalyst is in the range from 1-10 mol/100 (silicon+modifier metal) mol, more preferably, 2-8 mol/100 (silicon+modifier metal) mol, most preferably, 2.5-6 mol/100 (silicon+modifier metal) mol in the catalyst.

Alternatively, the catalyst may have a wt % of catalytic metal in the range 1 to 22 wt % in the catalyst, more preferably 4 to 18 wt %, most preferably, 5-13 wt %. These amounts would apply to all alkali metals, but especially caesium.

Accordingly, the catalytic metal:modifier metal mole ratio in the catalyst is typically at least 1.4 or 1.5:1, preferably, it is in the range 1.4 to 5:1 such as 1.5 to 4.0 :1, especially, 1.5 to 3.6 :1, typically in this regard the catalytic metal is caesium. Generally, herein, the catalytic metal is in excess of that which would be required to neutralise the modifier metal.

Preferably, the catalytic metal is present in the range 0.5-7.0 mol/mol modifier metal, more preferably 1.0-6.0 mol/mol, most preferably 1.5-5.0 mol/mol modifier metal.

### Calcination

It will be understood by the skilled person that a catalytic metal of the present invention may be added to the modified silica support by any suitable means. The catalytic metal is fixed, by calcination onto the support after deposition of the catalytic metal compound onto the support. The process of calcination is well known to those skilled in the art.

In preferred calcinations of the catalyst, the temperature is at least 450° C., more preferably, at least 475° C., most preferably, at least 500° C., especially, at least 600° C., more especially, above 700° C. Typically, the calcination temperature is in the range 400-1000° C., more typically, 500-900° C., most typically, 600-850° C.

The calcination atmosphere should typically contain some oxygen but may be an inert atmosphere or in vacuo, suitably 1-30% oxygen and most suitably 2-20% oxygen. The calcination time may typically be between 0.01 and 100 hours, suitably 0.5-40 hours, most suitably 1-24 hours.

### General Process

It will be understood by a skilled person that the catalytic metal may be added to the modified silica by any suitable means. Typically, in order to produce the modified silica catalyst, the modified silica is contacted with a catalytic metal.

Typically, in order to produce the catalyst, the modified silica support is contacted with an 100% aqueous solution of the catalytic metal or an acidic, neutral or alkaline aqueous solution containing a catalytic metal such as caesium, in the form of a salt of a catalytic metal and a base. Alternatively, the support can be contacted with a water miscible solution of the catalytic metal salt in an organic solvent. Preferred solvents are alcohols such as methanol, ethanol, propanol and isopropanol, preferably methanol. The most preferred solvent is methanol. Most preferably, the catalytic metal is added as a salt solution in methanol. Low levels of water, typically up to 20 vol % can be contained in the solutions.

Typically, the conditions of temperature, contact time and pH during this stage of the catalyst production process are such as to allow for impregnation of the modified silica support with the catalytic metal to form a modified silica supported catalyst.

Typical conditions of temperature for this step are between 5-95° C., more typically 10-80° C. and most typically between 20-70° C. The temperature for this step may be at least 5° C., more typically at least 10° C., most typically, at least 20° C.

Typical contact times between the modified support and the catalytic metal containing solution for this step may be between 0.05-48 hours, more typically between 0.1-24 hours, most typically between 0.5-18 hours. The contact time may be at least 0.05 hours, more typically at least 0.1 hours, most typically at least 0.5 hours.

The concentration of the catalytic metal salt solution for this step is dependent on a large number of factors including the solubility limit of the catalytic metal compound, the porosity of the modified silica support, the desired loading of the catalytic metal on the support and the method of addition, including the amount of liquid used to impregnate the support, the pH and the choice of the catalytic metal compound. The concentration in solution is best determined by experiment.

Suitable salts of catalytic metals for incorporation of the catalytic metal generally may be selected from one or more of the group consisting of formate, acetate, propionate, hydrogen carbonate, chloride, nitrate, hydroxide and carbonate, more typically, hydroxide, acetate or carbonate and most typically hydroxide and/or carbonate. The pH can be controlled during the impregnation by addition of ammonia with the metal compound or by using an appropriate catalytic metal compound such as the formate, carbonate, acetate or hydroxide, more preferably, the hydroxide or carbonate, in all cases either alone, in combination, or together with an appropriate carboxylic acid.

The control of the pH in the preferred ranges is most important at the end of the impregnation to effect satisfactory adsorption. Most typically, these salts may be incorporated using an alkaline solution of the salt. If the salt is not itself alkaline then a suitable base such as ammonium hydroxide may be added. As hydroxide salts are basic in nature, mixtures of one or more of the above salts with the hydroxide salt of the particular catalytic metal such as caesium may conveniently be prepared.

Addition of the catalytically active metal can be carried out by the method described above or can be by any other normal method used to impregnate catalyst supports, such as xerogel supports, such as using water or a solvent other than water such as an alcohol, suitably methanol, ethanol, propanol or isopropanol or using the incipient wetness method where only sufficient solution is added to the xerogel supports to fill the pores of the xerogel support. In this case, the concentration of the catalytically active metal may be calculated so as to introduce the target amount of catalytically active metal to the xerogel support material rather than providing an excess of solution of lower concentration. The addition of the catalytically active metal may utilise any preferred methodology known in the art.

The drying of the modified silica prior to calcination may take place in the temperature range of 20-200° C., more typically, 30-180° C., most typically, 40-150° C. The drying of the modified silica prior to calcination may take place at atmospheric or sub-atmospheric pressures, in the range of 0.001-1.01 bar. The drying of the modified silica may also be effected under a flow of inert gas in a static or fluidised bed. The drying times may be in the range between 0.1-24 hours, more typically between 0.5-12 hours, most typically between 1 and 6 hours.

Reduced pressure drying at lower temperatures or fluidised bed drying with an inert gas are suitable techniques.

### General Properties

The modifier metal and catalytic metal adsorbates in the final catalyst are generally metal oxide moieties.

### Modifier Metal

Typically, the modifier metal is present in the modified silica support in an effective amount to reduce sintering and improve selectivity of the catalyst. Typically, at least 30%, such as at least 35%, more preferably at least 40%, such as at least 45%, most suitably at least 50%, such as at least 55%, for example at least 60% or 65%, and most preferably at least 70% such as at least 75% or 80%, more typically, at least 85%, most typically, at least 90%, especially, at least 95% of modifier metal in the modified silica support is in mono- or dinuclear metal moieties, or is derived from a mono- or dinuclear modifier metal complex having one or more chelating ligands at the commencement of the modified silica formation at such levels.

Typically, the modifier metal is uniformly distributed throughout the support surface.

Preferably, the level of modifier metal present in the modified silica or catalyst may be up to 7.6×10⁻² mol/mol of silica, more preferably up to 5.9×10⁻² mol/mol of silica, most preferably up to 3.5×10⁻² mol/mol of silica. Typically, the level of such metal is between 0.067×10⁻² and 7.3×10⁻² mol/mol of silica, more preferably, between 0.13×10⁻² and 5.7×10⁻² mol/mol of silica and most preferably between 0.2×10⁻² and 3.5×10⁻² mol/mol of silica. Typically, the level of modifier metal present is at least 0.1×10⁻² mol/mol of silica, more preferably, at least 0.15×10⁻² mol/mol of silica and most preferably at least 0.25×10⁻² mol/mol of silica.

Preferably, the % w/w level of modifier metal will depend on the metal but may be up to 20% w/w of the modified silica support, more preferably up to 16% w/w, most preferably up to 11% w/w. Typically, the level of modifier metal is between 0.02-20% w/w of the modified silica support, more preferably between 0.1-15% w/w and most preferably between 0.15-10% w/w. Typically, the level of modifier metal is at least 0.02% w/w such as 0.25% w/w of the modified silica support, for example, 0.4% w/w, more typically, at least 0.5% w/w, most typically, at least 0.75% w/w.

### Catalyst

Typically, the catalyst of the invention may be in any suitable form. Typical embodiments are in the form of discrete particles. Typically, in use, the catalyst is in the form of a fixed bed of catalyst. Alternatively, the catalyst may be in the form of a fluidised bed of catalyst. A further alternative is a monolith reactor.

Where the catalysts are used in the form of a fixed bed, it is desirable that the supported catalyst is formed into granules, aggregates or shaped units, e.g. spheres, cylinders, rings, saddles, stars, poly-lobes prepared by pelleting, or extrusion, typically having maximum and minimum dimensions in the range 1 to 10 mm, more preferably, with a mean dimension of greater than 2mm such as greater than 2.5 or 3 mm. The catalysts are also effective in other forms, e.g. powders or small beads of the same dimensions as indicated. Where the catalysts are used in the form of a fluidised bed it is desirable that the catalyst particles have a maximum and minimum dimension in the range of 10-500 μm, preferably 20-200 μm, most preferably 20-100 μm.

The average pore volume of the catalyst particles may be less than 0.1 cm³/g but is generally in the range 0.1-5 cm³/g as measured by uptake of a fluid such as water. However, microporous catalysts with very low porosity are not the most preferred because they may inhibit movement of reagents through the catalyst and a more preferred average pore volume is between 0.2-2.0 cm³/g. The pore volume can alternatively be measured by a combination of nitrogen adsorption at 77 K and mercury porosimetry. The Micromeritics TriStar Surface Area and Porosity Analyser is used to determine pore volume as in the case of surface area measurements and the same standards are employed.

### Catalytic Process

According to a seventh aspect of the present invention there is provided a method of producing an ethylenically unsaturated carboxylic acid or ester, typically, an α, β ethylenically unsaturated carboxylic acid or ester, comprising the steps of contacting formaldehyde or a suitable source thereof with a carboxylic acid or ester in the presence of catalyst and optionally in the presence of an alcohol, wherein the catalyst is according to any of the other aspects of the present invention defined herein.

Advantageously, it has also been found that catalysts comprising modified silicas as defined herein and containing a catalytic metal are remarkably effective catalysts for the production of α, β ethylenically unsaturated carboxylic acid or esters by condensation of the corresponding acid or ester with a methylene source such as formaldehyde having reduced sintering of the catalyst surface, improved selectivity and providing high catalyst surface area. In particular enhanced properties are found when the modified silica support is uncalcined prior to treatment with the catalytic metal. Furthermore, the use of certain metal complexes to incorporate the modifier metal onto the support by adsorption provides a more dispersed distribution of mono- or dinuclear modifier metal moieties.

By the term “a suitable source thereof” in relation to formaldehyde herein is meant that the free formaldehyde may either form in situ from the source under reaction conditions or that the source may act as the equivalent of free formaldehyde under reaction conditions, for example it may form the same reactive intermediate as formaldehyde so that the equivalent reaction takes place.

A suitable source of formaldehyde may be a compound of formula (I):

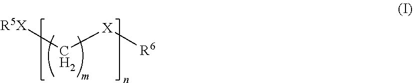

wherein R⁵ and R⁶ are independently selected from C₁-C₁₂ hydrocarbons or H, X is O, n is an integer from 1 to 100, and m is 1.

Typically, R⁵ and R⁶ are independently selected from C₁-C₁₂ alkyl, alkenyl or aryl as defined herein, or H, more suitably, C₁-C₁₀ alkyl, or H, most suitably, C₁-C₆ alkyl or H, especially, methyl or H. Typically, n is an integer from 1 to 10, more suitably 1 to 5, especially, 1-3.

However, other sources of formaldehyde may be used including trioxane.

Therefore, a suitable source of formaldehyde also includes any equilibrium composition which may provide a source of formaldehyde. Examples of such include but are not restricted to dimethoxymethane, trioxane, polyoxymethylenes R¹—O—(CH₂—O)ᵢ—R² wherein R¹ and/or R² are alkyl groups or hydrogen, i=1 to 100, paraformaldehyde, formalin (formaldehyde, methanol, water) and other equilibrium compositions such as a mixture of formaldehyde, methanol and methyl propionate.

Polyoxymethylenes are higher formals or hemiformals of formaldehyde and methanol CH₃—O—(CH₂—O)ᵢ—CH₃ (“formal-i”) or CH₃—O—(CH₂—O)ᵢ—H (“hemiformal-i”), wherein i=1 to 100, suitably, 1-5, especially 1-3, or other polyoxymethylenes with at least one non methyl terminal group. Therefore, the source of formaldehyde may also be a polyoxymethylene of formula R³¹—O—(CH2-O—)ᵢ—R³², where R³¹ and R³² may be the same or different groups and at least one is selected from a C₁-C₁₀ alkyl group, for instance R³¹=isobutyl and R³²=methyl.

Generally, the suitable source of formaldehyde is selected from dimethoxymethane, lower hemiformals of formaldehyde and methanol, CH₃—O—(CH₂—O)ᵢ—H where i=1-3, formalin or a mixture comprising formaldehyde, methanol and methyl propionate.

Typically, by the term formalin is meant a mixture of formaldehyde:methanol:water in the ratio 25 to 65%: 0.01 to 25%: 25 to 70% by weight. More typically, by the term formalin is meant a mixture of formaldehyde:methanol:water in the ratio 30 to 60%:

0.03 to 20%: 35 to 60% by weight. Most typically, by the term formalin is meant a mixture of formaldehyde:methanol:water in the ratio 35 to 55%: 0.05 to 18%: 42 to 53% by weight.

Typically, the mixture comprising formaldehyde, methanol and methyl propionate contains less than 5% water by weight. More suitably, the mixture comprising formaldehyde, methanol and methyl propionate contains less than 1% water by weight. Most suitably, the mixture comprising formaldehyde, methanol and methyl propionate contains 0.1 to 0.5% water by weight.

According to an eighth aspect of the present invention, there is provided a process for preparing an ethylenically unsaturated acid or ester comprising contacting an alkanoic acid or ester of the formula R¹-CH₂-COOR³, with formaldehyde or a suitable source of formaldehyde of formula (I) as defined below:

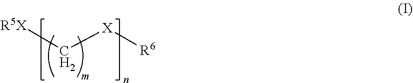

where R5 is methyl and R6 is H;

### X is O;

m is 1;
and n is any value between 1 and 20 or any mixture of these;
in the presence of a catalyst according to any aspect of the present invention, and optionally in the presence of an alkanol; wherein R1 is hydrogen or an alkyl group with 1 to 12, more Suitably, 1 to 8, most suitably, 1 to 4 carbon atoms and R3 may also be independently, hydrogen or an alkyl group with 1 to 12, more suitably, 1 to 8, most suitably, 1 to 4 carbon atoms.

Therefore, the present inventors have discovered that producing catalyst according to the present invention enables surprising improvement in selectivity for the condensation of methylene sources such as formaldehyde with a carboxylic acid or alkyl ester such as methyl propionate to form ethylenically unsaturated carboxylic acids. In addition, the rate of sintering of the catalyst surface during the condensation reaction is significantly and surprisingly reduced.

Accordingly, one particular process for which the catalysts of the present invention have been found to be particularly advantageous is the condensation of formaldehyde with methyl propionate in the presence of methanol to produce MMA.

In the case of production of MMA, the catalyst is typically contacted with a mixture comprising formaldehyde, methanol and methyl propionate.

The process of the seventh or eighth aspect of the invention is particularly suitable for the production of acrylic and alkacrylic acids and their alkyl esters, and also methylene substituted lactones. Suitable methylene substituted lactones include 2-methylene valerolactone and 2-methylene butyrolactone from valerolactone and butyrolactone respectively. Suitable, (alk)acrylic acids and their esters are (C₀₋₈alk)acrylic acid or alkyl (C₀₋₈alk)acrylates, typically from the reaction of the corresponding alkanoic acid or ester thereof with a methylene source such as formaldehyde in the presence of the catalyst, suitably the production of methacrylic acid, acrylic acid, methyl methacrylate, ethyl acrylate or butyl acrylate, more suitably, methacrylic acid or especially methyl methacrylate(MMA) from propanoic acid or methyl propionate respectively. Accordingly, in the production of methyl methacrylate or methacrylic acid, the preferred ester or acid of formula R¹—CH₂—COOR³ is methyl propionate or propionic acid respectively and the preferred alkanol is therefore methanol. However, it will be appreciated that in the production of other ethylenically unsaturated acids or esters, the preferred alkanols or acids will be different.

The reaction of the present invention may be a batch, semi-batch or continuous reaction.

Typical conditions of temperature and gauge pressure in the process of the seventh or eighth aspect of the invention are between 100° C. and 400° C., more preferably, 200° C. and 375° C., most preferably, 275° C. and 360° C.; and/or between 0.001 MPa and 1 MPa, more preferably between 0.03 MPa and 0.5 MPa, most preferably between 0.03 MPa and 0.3 MPa. Typical residence times for the reactants in the presence of the catalyst are between 0.1 and 300 secs, more preferably between, 1-100 secs, most preferably between 2-50 secs, especially, 3-30 secs.

The amount of catalyst used in the process of production of product in the present invention is not necessarily critical and will be determined by the practicalities of the process in which it is employed. However, the amount of catalyst will generally be chosen to affect the optimum selectivity and yield of product and an acceptable temperature of operation. Nevertheless, the skilled person will appreciate that the minimum amount of catalyst should be sufficient to bring about effective catalyst surface contact of the reactants. In addition, the skilled person would appreciate that there would not really be an upper limit to the amount of catalyst relative to the reactants but that in practice this may be governed again by the contact time required and/or economic considerations.

The relative amount of reagents in the process of the seventh or eighth aspect of the invention can vary within wide limits but generally the mole ratio of formaldehyde or suitable source thereof to the carboxylic acid or ester is within the range of 20:1 to 1:20, more suitably, 5:1 to 1:15. The most preferred ratio will depend on the form of the formaldehyde and the ability of the catalyst to liberate formaldehyde from the formaldehydic species. Thus highly reactive formaldehydic substances where one or both of R³¹ and R³² in R³¹O—(CH₂—O)ᵢR³² is H require relatively low ratios, typically, in this case, the mole ratio of formaldehyde or suitable source thereof to the carboxylic acid or ester is within the range of 1:1 to 1:9. Where neither of R³¹ and R³² is H, as for instance in CH₃O—CH₂—OCH₃, or in trioxane higher ratios are most preferred, typically, 6:1 to 1:3.

As mentioned above, due to the source of formaldehyde, water may also be present in the reaction mixture. Depending on the source of formaldehyde, it may be necessary to remove some or all of the water therefrom prior to catalysis. Maintaining lower levels of water than that in the source of formaldehyde may be advantageous to the catalytic efficiency and/or subsequent purification of the products. Water at less than 10 mole % in the reactor is preferred, more suitably, less than 5 mole %, most suitably, less than 2 mole %.

The molar ratio of alcohol to the acid or ester is typically within the range 20:1 to 1:20, preferably 10:1 to 1:10, most preferably 5:1 to 1:5, for example 1:1.5. However, the most preferred ratio will depend on the amount of water fed to the catalyst in the reactants plus the amount produced by the reaction, so that the preferred molar ratio of the alcohol to the total water in the reaction will be at least 1:1 and more preferably at least 2:1.

The reagents of the seventh or eighth aspect may be fed to the reactor independently or after prior mixing and the process of reaction may be continuous or batch. Typically, however, a continuous process is used.

Typically, the method of the seventh or eighth aspect of the present invention is carried out when reactants are in the gaseous phase.

In a still further aspect, the invention extends to the process of producing an ethylenically unsaturated carboxylic acid or ester according to any of the relevant aspects herein comprising the steps of first producing a catalyst according to any of the relevant aspects herein.

### DEFINITIONS

By uncalcined modified silica support is meant that the silica support is not calcined (such as by treatment above 275° C. or 325° C. or 375° C. or 425° C.) after the modification step and before treatment with the catalytic metal and does not necessarily mean that the original silica support is uncalcined prior to modification by the modifier metal. Similarly, by uncalcined catalyst intermediate is meant that the modified silica support is uncalcined since its modification and does not necessarily mean that the original unmodified silica support is uncalcined prior to modification by the modifier metal.

By the term “impregnated” as used herein is included the addition of the catalytic metal dissolved in a solvent, to make a solution, which is added to the xerogel or aerogel, such that the solution is taken up into the voidages within the said xerogel or aerogel. The term also extends to replacing a hydrogel liquid with a suitable solvent and adding the catalytic metal as a solution in the solvent to effect mass transfer into the hydrogel by diffusion.

The silica support may be treated by the mononuclear and/or dinuclear modifier metal by any of the various techniques known to those skilled in the art of support formation. The silica support may be contacted with the mononuclear or dinuclear modifier metal in such a manner so as to disperse modifier metal throughout the silica support. Typically, the modifier metal may be uniformly distributed throughout the surface of the silica support. Preferably, the modifier metal is dispersed through the silica support by adsorption.

By the term “adsorption” or the like in relation to the modifier metal or catalytic metal as used herein is meant the incorporation thereof onto the silica support surface by the interaction thereof with the silica support, optionally by physisorption but typically by chemisorption. Typically, addition of the modifier to the silica support involves the steps of: adsorption of the metal cation source onto the silica support to form a metal complex residue and drying of the support to convert the metal complexes to metal oxide moieties. Typically, there is therefore a random distribution of modifier metal throughout the silica support contacted.

For the avoidance of doubt, modifier metal moieties having a total of 1 metal atom are considered mononuclear. It will be appreciated that in a silica network the modifier metal moieties are associated with the silica network and therefore the term mono- or dinuclear moiety is a reference to the modifier metal and its immediately surrounding atoms and not to the silicon atoms of the network or to other modifier metal atoms associated with the network but nevertheless forming part of separate generally unassociated moieties.

Modifier metal and modifier metal oxide moieties in the modified silica support according to the present invention relate to modifier metal, not to silicon or silica. Similarly, the modifier metal herein is not the same metal as the catalytic metal.

Unless indicated to the contrary, amounts of modifier or catalytic metal or modifier or catalytic metal in the catalyst relate to the modifier or catalytic metal ion and not the surrounding atoms.

Levels of catalytic metal in the catalyst whether moles, wt % or otherwise may be determined by appropriate sampling and taking an average of such samples. Typically, 5-10 samples of a particular catalyst batch would be taken and alkali metal levels determined and averaged, for example by XRF, atomic absorption spectroscopy, neutron activation analysis, ion coupled plasma mass spectrometry (ICPMS) analysis or ion coupled plasma atomic emission spectroscope (ICPAES).

Levels of the metal oxide of particular types in the catalyst/support are determined by XRF, atomic absorption spectroscopy, neutron activation analysis or ion coupled plasma mass spectrometry (ICPMS) analysis.

The typical average surface area of the modified silica supported catalyst according to any aspect of the invention is in the range 20-600 m²/g, more preferably 30-450 m²/g and most preferably 35-350 m²/g as measured by the B.E.T. multipoint method using a Micromeritics Tristar 3000 Surface Area and porosity analyser. The reference material used for checking the instrument performance may be a carbon black powder supplied by Micromeritics with a surface area of 30.6 m²/g (+/−0.75 m²/g), part number 004-16833-00.)

The term “alkyl” when used herein, means, unless otherwise specified, C₁ to C12 alkyl and includes methyl, ethyl, ethenyl, propyl, propenyl butyl, butenyl, pentyl, pentenyl, hexyl, hexenyl and heptyl groups, typically, the alkyl groups are selected from methyl, ethyl, propyl, butyl, pentyl and hexyl, more typically, methyl. Unless otherwise specified, alkyl groups may, when there is a sufficient number of carbon atoms, be linear or branched, be cyclic, acyclic or part cyclic/acyclic, be unsubstituted, substituted or terminated by one or more substituents selected from halo, cyano, nitro, —OR¹⁹, —OC(O)R²⁰, —C(O)R²¹, —C(O)OR²², —NR²³R²⁴, -C(0)NR²⁵R²⁶, —SR²⁹, —C(O)SR³⁰, —C(S)NR²⁷R²⁸, unsubstituted or substituted aryl, or unsubstituted or substituted Het, wherein R¹⁹ to R³⁰ here and generally herein each independently represent hydrogen, halo, unsubstituted or substituted aryl or unsubstituted or substituted alkyl, or, in the case of R²¹, halo, nitro, cyano and amino and/or be interrupted by one or more (typically less than 4) oxygen, sulphur, silicon atoms, or by silano or dialkylsilcon groups, or mixtures thereof. Typically, the alkyl groups are unsubstituted, typically, linear and typically, saturated.

The term “alkenyl” should be understood as “alkyl” above except at least one carbon-carbon bond therein is unsaturated and accordingly the term relates to C2 to C12 alkenyl groups.

The term “alk” or the like should, in the absence of information to the contrary, be taken to be in accordance with the above definition of “alkyl” except “Co alk” means non-substituted with an alkyl.

The term “aryl” when used herein includes five-to-ten-membered, typically five to eight membered, carbocyclic aromatic or pseudo aromatic groups, such as phenyl, cyclopentadienyl and indenyl anions and naphthyl, which groups may be unsubstituted or substituted with one or more substituents selected from unsubstituted or substituted aryl, alkyl (which group may itself be unsubstituted or substituted or terminated as defined herein), Het (which group may itself be unsubstituted or substituted or terminated as defined herein), halo, cyano, nitro, OR¹⁹, OC(O)R²⁰, C(O)R21, C(O)OR²², NR²³R²⁴, C(O)NR²⁵R²⁶, SR²⁹, C(O)SR³⁰ or C(S)NR²⁷R²⁸ wherein R¹⁹ to R³⁰ each independently represent hydrogen, unsubstituted or substituted aryl or alkyl (which alkyl group may itself be unsubstituted or substituted or terminated as defined herein), or, in the case of R²¹, halo, nitro, cyano or amino.

The term “halo” when used herein means a chloro, bromo, iodo or fluoro group, typically, chloro or fluoro.

The term “Het”, when used herein, includes four- to twelve-membered, typically four- to ten-membered ring systems, which rings contain one or more heteroatoms selected from nitrogen, oxygen, sulfur and mixtures thereof, and which rings contain no, one or more double bonds or may be non-aromatic, partly aromatic or wholly aromatic in character. The ring systems may be monocyclic, bicyclic or fused. Each “Het” group identified herein may be unsubstituted or substituted by one or more substituents selected from halo, cyano, nitro, oxo, alkyl (which alkyl group may itself be unsubstituted or substituted or terminated as defined herein) 'OR¹⁹, —OC(O)R²⁰, —C(O)R²¹, —C(O)OR²², —N(R²³)R²⁴, —C(O)N(R²⁵)R²⁶, —SR²⁹, —C(O )SR³⁰ or —C(S)N(R27)R²⁸ wherein R¹⁹ to R³⁰ each independently represent hydrogen, unsubstituted or substituted aryl or alkyl (which alkyl group itself may be unsubstituted or substituted or terminated as defined herein) or, in the case of R²¹, halo, nitro, amino or cyano. The term “Het” thus includes groups such as optionally substituted azetidinyl, pyrrolidinyl, imidazolyl, indolyl, furanyl, oxazolyl, isoxazolyl, oxadiazolyl, thiazolyl, thiadiazolyl, triazolyl, oxatriazolyl, thiatriazolyl, pyridazinyl, morpholinyl, pyrimidinyl, pyrazinyl, quinolinyl, isoquinolinyl, piperidinyl, pyrazolyl and piperazinyl. Substitution at Het may be at a carbon atom of the Het ring or, where appropriate, at one or more of the heteroatoms.

“Het” groups may also be in the form of an N oxide.

Suitable optional alcohols for use in the catalysed reaction of the seventh and eighth aspects of the present invention may be selected from: a C₁-C₃₀ alkanol, including aryl alcohols, which may be optionally substituted with one or more substituents selected from alkyl, aryl, Het, halo, cyano, nitro, OR¹⁹, OC(O)R²⁰, C(O)R²¹, C(O)OR²², NR²³R²⁴, C(O)NR²⁵R²⁶, C(S)NR²⁷R²⁸, SR²⁹ or C(O)SR³⁰ as defined herein. Highly preferred alkanols are C₁-C₈ alkanols such as methanol, ethanol, propanol, iso-propanol, iso-butanol, t-butyl alcohol, phenol, n-butanol and chlorocapryl alcohol, especially, methanol. Although the monoalkanols are most preferred, poly-alkanols, typically, selected from di-octa ols such as diols, triols, tetra-ols and sugars may also be utilised. Typically, such polyalkanols are selected from 1, 2-ethanediol, 1,3-propanediol, glycerol, 1,2,4 butanetriol, 2-(hydroxymethyl)-1,3-propanediol, 1,2,6 trihydroxyhexane, pentaerythritol, 1,1,1 tri(hydroxymethyl)ethane, nannose, sorbase, galactose and other sugars. Preferred sugars include sucrose, fructose and glucose. Especially preferred alkanols are methanol and ethanol. The most preferred alkanol is methanol. The amount of alcohol is not critical. Generally, amounts are used in excess of the amount of substrate to be esterified. Thus, the alcohol may serve as the reaction solvent as well, although, if desired, separate or further solvents may also be used.

The term “gel” as used herein is also known to the skilled person but in case of doubt may be taken to be a solid network in which a fluid is dispersed. Generally, the gel is a polymer network in which fluid is dispersed. A co-gel is a term used to indicate that more than one original chemical compound/moiety is incorporated into the polymeric network, usually silica and a metal oxide or salt. Accordingly, co-gelation herein means the formation of a co-gel.

A gel is thus a sol that has set. A Hydrogel is thus a gel as defined herein where the fluid is water. A Xerogel is a gel that has been dried to remove the fluid. An Aerogel is a gel in which the fluid is replaced by a gas and therefore is not subject to the same shrinkage as a Xerogel.

The term commencement herein means the beginning of the formation of the modified silica.

The term “moieties” as used herein in relation to the modifier metal is used to refer to the form of the modifier metal on the modified support. Although, the adsorbed modifier metal generally forms part of a network, the modifier metal will be in the form of discrete residues on the silica substrate whether as a metal complex or oxide and whether, in the latter case, before or after calcination. The term mononuclear means having a single metal centre and in the case of moieties on the silica means having the form of a mononuclear residue. Dinuclear should be interpreted accordingly.

% of the modifier metal has no units herein because it refers to number of metal atoms per total number of such atoms. It will be appreciated that the moieties may take the form of non-mono or dinuclear clusters but that these clusters are still made up of modifier metal atoms.

The term “surface” as used herein in relation to the silica support, unless stated otherwise, includes the surface of the silica within the pores of the silica, more particularly, within the macro- and mesopores thereof.

Embodiments of the invention will now be defined by reference to the accompanying examples in which:

### EXPERIMENTAL

### Silica Support Description

### Example 1 (Preparative)

Fuji Silysia CARiACT Q10 silica was dried in a laboratory oven at 160° C. for 16 hours, after which it was removed from the oven and cooled to room temperature in a sealed flask stored in a desiccator. This silica had a surface area of 333 m²/g, a pore volume of 1.0 ml/g, and an average pore diameter of 10 nm as determined by nitrogen adsorption/desorption isotherm analysis (Micromeritics Tristar II). This silica is primarily composed of spherical silica beads in the diameter range of 2.0-4.0 mm.

### Zr Modification of Silica Supports

### Example 2 (2.7 wt % Zr, Comparative)

1.671 g of, Zr(acac)4 (97%, Sigma Aldrich) was dissolved in 20 ml of MeOH (99% Sigma Aldrich). In a separate flask 10 g of the silica from Example 1 was weighed off. The weighed off silica was then added to the Zr(acac)₄ solution with agitation. Agitation was continued until the pore volume of the silica was completely occupied by solvent effectively forming a slurry. Once pore filling had been completed the Zr-modified silica was left for 16 hours in a sealed flask with periodic agitation. After this time the extra-porous solution was removed by filtration. This was followed by a drying step where the intra-porous organic solvent was removed by passing a flow of nitrogen gas over the wet Zr-modified silica at room temperature. Alternatively, the intra-porous solvent was removed on a rotary evaporator at reduced pressure. Once all the solvent had been removed the Zr-modified silica support was calcined in a furnace at 500 ° C. under a flow of air with a heating ramp rate of 5 ° C./min and a final hold of 5 hours. Upon cooling this yielded the Zr grafted silica support with an 89% Zr usage efficiency. The Zr load (wt %) on the Zr-modified support was determined via powder Energy Dispersive X-Ray Fluorescence analysis (Oxford Instruments X-Supreme8000).

### Example 3 (2.7 wt % Zr)

A support modification as described in Example 2 was performed except that after the drying step had been completed an additional 16 h drying step in a laboratory oven set at 110-120° C. was performed. Additionally, the high temperature calcination step at 500° C. was not performed. This yielded a Zr grafted silica support with an 89% Zr usage efficiency. (Note: the Zr loading was determined after an oxidative calcination at 500° C. of a sample of the Zr grafted material).

### Cs Modification of Modified Supports

### Example 4 (11.3 wt % Cs, 2.4 wt % Zr, Comparative)

1.80 g of CsOH.H₂O (99.5% Sigma Aldrich) was weighed out in a glovebox and dissolved in 20 ml of a 9:1 v/v MeOH:H₂O solvent mixture. 10 g of the modified silica from Example 2 was added to the CsOH solution with agitation. Agitation was continued for an additional 15 min after which the sample was left for 16 hours in a sealed flask with periodic agitation. After this time the extra-porous solution was removed by filtration. This was followed by a drying step where the intra-porous solvent was removed by passing a flow of nitrogen gas over the wet Cs/Zr-modified silica at room temperature. Alternatively, the intra-porous solvent was removed on a rotary evaporator at reduced pressure. Following this the catalyst beads were placed into a drying oven at 110-120° C. and left to dry for 16 hours. Upon cooling this yielded the Cs/Zr/SiO₂ catalyst with a 90% Cs usage efficiency. The Cs load (wt %) on the catalyst was determined via powder Energy Dispersive X-Ray Fluorescence analysis (Oxford Instruments X-Supreme8000).

### Example 5 (11.0 wt % Cs, 2.4 wt % Zr, Comparative)

A catalyst was prepared as described in Example 4 except that 1.75 g of CsOH.H₂O was used. Additionally, after the drying step at 120° C. the catalyst was calcined in a furnace at 700° C. under a flow of air with a heating ramp rate of 5° C./min and a final hold of 5 hours. Upon cooling this yielded the Cs/Zr/SiO₂ catalyst.

### Example 6 (11.3 wt % Cs, 2.4 wt % Zr)

A catalyst was prepared as described in Example 4 except that 10.5 g of silica from Example 3 was used. Additionally, after the drying step at 120° C. the catalyst was calcined in a furnace at 700° C. under a flow of air with a heating ramp rate of 5° C./min and a final hold of 5 hours. Upon cooling this yielded the Cs/Zr/SiO₂ catalyst.

### Example 7 (10.6 wt % Cs, 2.4 wt % Zr)

A catalyst was prepared as described in Example 4 except that 10.5 g of silica from Example 3 was used and water was used as a solvent instead of 9:1 v/v MeOH:H₂O. Additionally, after the drying step at 120° C. the catalyst was calcined in a furnace at 400° C. under a flow of air with a heating ramp rate of 5° C./min and a final hold of 5 hours. Upon cooling this yielded the Cs/Zr/SiO₂ catalyst.

### Example 8 (10.6 wt % Cs, 2.4 wt % Zr)

A catalyst was prepared as described in Example 7 except that final calcination was performed at 600° C.

### Example 9 (10.6 wt % Cs, 2.4 wt % Zr)

A catalyst was prepared as described in Example 7 except that final calcination was performed at 700° C.

### Example 10 (Catalytic Performance Testing)

Catalysts from Example 4 to Example 9 were tested for the reaction of methyl propionate and formaldehyde in a labscale microreactor. For this, 3 g of catalyst was loaded into a fixed bed reactor with an internal tube diameter of 10 mm. The reactor was heated to 330° C. and preconditioning was performed by feeding a vaporised stream comprising of 70 wt % methyl propionate, 20 wt % methanol, 6 wt % water and 4 wt % formaldehyde from a vaporiser fed by a Gilson pump at 0.032 ml/min. This preconditioning was continued overnight. After preconditioning a feed stream comprising of 75.6 wt % methyl propionate, 18.1 wt % methanol, 5.7 wt % formaldehyde and 0.6 wt % water, was pumped by a Gilson pump to a vaporiser set at 330° C. before being fed to the heated reactor set at 330° C. containing the catalyst. The reactor exit vapour was cooled and condensed with samples being collected at five different liquid feed rates (between 0.64-0.032 ml/min) so as to obtain conversions at varying vapour/catalyst contact times. The liquid feed and condensed ex-reactor liquid products were analysed by a Shimadzu 2010 Gas Chromatograph with a DB1701 column. The compositions of the samples were determined from the respective chromatograms and yields and selectivities at varying contact times determined. Activity was defined as the inverse of the contact time, in seconds, required to obtain 12% MMA+MAA yield on methyl propionate fed and was determined via an interpolation on a contact time vs. MMA+MAA yield graph. This interpolated contact time was then used to obtain the MMA+MAA selectivity at 12% MMA+MAA yield.

TABLE 1
Activity and MMA + MAA selectivity results for catalysts prepared
according to Example 4 to Example 9 and tested according to Example 10.

| | | | Cs:Zr | | Activity at 12% | |
|---|---|---|---|---|---|---|
| | Zr load | Cs load | (molar | Catalyst calcination | MMA + MAA yield | MMA + MAA |
| Example | (wt %) | (wt %) | ratio) | temperature (° C.) | (1/s) | selectivity (%) |
| Example 4 (comp) | 2.4 | 11.3 | 3.2 | None | 0.51 | 96.1 |
| Example 5 (comp) | 2.4 | 11.0 | 3.1 | 700 | 0.61 | 96.1 |
| Example 6 | 2.4 | 11.3 | 3.2 | 700 | 0.49 | 97.5 |
| Example 7 | 2.4 | 10.6 | 3.0 | 400 | 0.44 | 94.5 |
| Example 8 | 2.4 | 10.6 | 3.0 | 600 | 0.49 | 96.4 |
| Example 9 | 2.4 | 10.6 | 3.0 | 700 | 0.52 | 97.5 |

### Example 11 (Catalyst Stability Determination)

Initial catalyst stability was assessed by measurement of the surface area (nitrogen adsorption/desorption isotherm analysis, Micromeritics Tristar II) after a calcination treatment at 700° C. according to Example 5. This provided a means to assess the surface stabilisation imparted to the catalyst.

TABLE 2
Surface area of catalysts subjected to a 700° C.
calcination treatment as a measure of initial stabilisation.

| | Surface area after 700° C. |
|---|---|
| Example | calcination (m²/g) |
| Example 4 (comp) | 118 |
| Example 5 (comp) | 156 |
| Example 6 | 210 |

### Example 12 (Accelerated Ageing Test)

Catalyst sintering resistance was assessed in an accelerated ageing test. For this, 1 g of catalyst was loaded into a U-tube stainless steel reactor and loaded into an oven.

The oven was heated to 385° C. and a stream of nitrogen (10 ml/min) was passed through a saturating vaporiser containing water that was heated to 92° C. This ensured that a feed stream with a water partial pressure of 0.75 bara was passed over the catalyst heated to 385° C. Periodically the surface area of the catalyst samples was determined ex-situ using nitrogen adsorption/desorption isotherm analysis (Micromeritics Tristar II).

TABLE 3
Accelerated ageing data for catalysts prepared according to
Example 4 to Example 8 and tested according to Example 12.

Surface area (m²/g) at time (days)

| Example | 0 | 1 | 7 | 14 | 21 | 28 |
|---|---|---|---|---|---|---|
| Example 4 | 229 | 179 | 162 | 149 | 151 | 154 |
| (comp) | | | | | | |
| Example 5 | 156 | 140 | 136 | 132 | 134 | 129 |
| (comp) | | | | | | |
| Example 6 | 210 | 200 | 203 | 203 | 194 | 187 |
| Example 7 | 258 | 258 | 192 | 202 | 199 | 199 |
| Example 8 | 242 | 224 | 208 | 197 | 200 | 200 |

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the preferred, typical or optional invention features disclosed in this specification (including any accompanying claims, abstract or drawings), or to any novel one, or any novel combination, of the preferred, typical or optional invention steps of any method or process so disclosed.

1. A process for producing a catalyst comprising the steps of:
a) providing an uncalcined metal modified porous silica support wherein the modifier metal is selected from one or more of boron, magnesium, aluminium, zirconium, hafnium and titanium, and wherein the modifier metal is present in mono- or dinuclear modifier metal moieties
b) optionally removing any solvent or liquid carrier from the modified silica support;
c) optionally drying the modified silica support;
d) treating the uncalcined metal modified silica support with a catalytic metal to effect adsorption of the catalytic metal onto the metal modified silica support; and
e) calcining the impregnated silica support of step d).

2. (canceled)

3. A method of producing a catalyst comprising the steps of:
a) providing a porous silica support having isolated silanol groups;
b) treating the said porous silica support with mono- or dinuclear modifier metal compound so that modifier metal is adsorbed onto the surface of the silica support through reaction with said isolated silanol groups, wherein the adsorbed modifier metal atoms are sufficiently spaced apart from each other to substantially prevent oligomerisation thereof with neighbouring modifier metal atoms prior to and preferably after calcination, more preferably, sufficiently spaced apart from each other to substantially prevent dimerisation or trimerisation thereof with neighbouring modifier metal atoms thereof wherein the modifier metal is selected from boron, magnesium, aluminium, zirconium, hafnium and titanium;
c) optionally removing any solvent or liquid carrier from the modified silica support;
d) optionally drying the modified silica support;
e) treating the uncalcined modified silica support with a catalytic metal to effect adsorption of the catalytic metal onto the modified silica support; and
f) calcining the impregnated silica support of step e).

4. The process according to claim 1:
wherein the porous silica support modified with a modifier metal is a modifier metal oxide-silica co-gel support.

5. (canceled)

6. (canceled)

7. The process according to claim 1, wherein the calcination step is carried out at a temperature of at

8. (canceled)

9. (canceled)

10. The process according to claim 1, wherein the silica support is a hydrogel or xerogel.

11. (canceled)

12. (canceled)

13. (canceled)

14. (canceled)

15. The process according to claim 1, wherein the modifier metal is an adsorbate adsorbed on the silica support surface.

16. (canceled)

17. The process according to claim 1, wherein the modifier metal is selected from zirconium, hafnium or titanium.

18. The process according to claim 1, wherein the catalytic metal is an alkali metal.

19. The process according to claim 1, wherein the silica support comprises the said modifier metal at a level of <5 metal atoms per nm².

20. The process according to claim1 , wherein at least 25%, of the said modifier metal on the support either before or after catalytic metal calcination is present in the form of mono- or dinuclear modifier metal moieties.

21. The process according to claim 1, wherein the adsorbed or co-gelated modifier metal cations are sufficiently spaced apart from each other to substantially prevent oligomerisation thereof during subsequent treatment steps such as the impregnation of catalytic metal and/or, calcination.

22. The process according to claim 1, wherein the silica support comprises isolated silanol groups (—SiOH) at a level of <2.5 groups per nm².

23. (canceled)

24. (canceled)

25. The process according to claim 1, wherein the support comprises the said modifier metal moieties at a level of >0.025 and <2.5 groups per nm².

26. (canceled)

27. The process according to claim 1, wherein the silica component of the modified silica support may typically form 80-99.9 wt % of the modified support.

28. The process according to claim1 , wherein the silica support has an average pore size of between 2 and 1000 nm.

29. The process according to claim 1, wherein the catalytic metal is an adsorbate adsorbed on the modified silica support surface of the catalyst.

30. The process according to claim1, wherein the catalytic metals such as caesium may be present in the catalyst at a level of at least 1 mol/100 (silicon+modifier metal) mol.

31. The process according to claim 1, the catalytic metal:modifier metal mole ratio in the catalyst is in the range 1.4 to 5:1.

32. The process according to claim 1, wherein, the catalytic metal is present in the range 0.5-7.0 mol.

33. The process according to claim 1, wherein the level of catalytic metal in the catalyst is in the range from 1-10 mol/100 (silicon+modifier metal) mol.

34. The process according to claim 1, wherein, the level of modifier metal present in the modified silica or catalyst may be up to 7.6×10⁻² mol/mol of silica.

35. The process according to claim 1, wherein, the level of modifier metal is between 0.067×10⁻² and 7.3×10⁻² mol/mol of silica.

36. The process according to claim 1, wherein, the level of modifier metal present is at least 0.1×10⁻² mol/mol of silica.

37. The process according to claim 1, wherein the average pore volume of the catalyst particles may be less than 0.1 cm³/g but is generally in the range 0.1-5 cm³/g as measured by uptake of a fluid such as water.

38. The process according to claim 1, wherein average pore volume of the catalyst is between 0.2-2.0 cm³/g.

39. (canceled)

40. (canceled)

41. (canceled)

42. (canceled)

43. (canceled)

44. The process according to claim1 , wherein the moieties or compounds are mononuclear.

45. The process according to claim 1, wherein the moieties are uniformly distributed throughout the surface of the silica support.

46. The process according to claim1, wherein the modifier metal compounds are uniformly distributed throughout the surface of the silica support.
